(19) United States
(12) Patent Application Publication
(10) Pub. No.: US 2022/0125360 A1
(43) Pub. Date: Apr. 28, 2022

(54) METHOD AND COMPUTER PROGRAM FOR DETERMINING PSYCHOLOGICAL STATE THROUGH DRAWING PROCESS OF COUNSELING RECIPIENT

(71) Applicant: RFCAMP LTD., Seoul (KR)

(72) Inventor: Kwon Soo KIM, Seoul (KR)

(73) Assignee: RFCAMP LTD., Seoul (KR)

(21) Appl. No.: 17/571,512

(22) Filed: Jan. 9, 2022

Related U.S. Application Data
(63) Continuation of application No. PCT/KR2020/008725, filed on Jul. 3, 2020.

(30) Foreign Application Priority Data
Sep. 4, 2019 (KR) 10-2019-0109531

(51) Int. Cl. A61B 5/16; G16H 50/20; G16H 70/20

(52) U.S. Cl. CPC A61B 5/165; G16H 70/20; G16H 50/20; A61B 5/167

(57) ABSTRACT

The present disclosure provides an electronic device for performing psychological counseling through pictures. The present disclosure also provides extracting detailed attribute information about a drawing process of a counseling recipient, and determining a psychological state of the counseling recipient in consideration of the detailed attribute information obtained during the drawing process in addition to a final image obtained through drawing. The present disclosure also provides transferring a final image, which is input data representing a psychological state of a counseling recipient, and detailed attribute information obtained during a drawing process, to a psychological analysis expert group and obtaining detailed analysis comments.

### TECHNICAL FIELD

The present specification relates to a method and a computer program for determining a psychological state through a drawing process of a counseling recipient.

### BACKGROUND ART

Various methods have been devised to more objectively analyze and classify complex human psychology. The various methods include personality test methods using multiple-choice items such as Minnesota Multiphasic Personality Inventory (MMPI), Myers-Briggs Type Indicator (MBTI), etc., test methods using pictures such as a Rorschach test, mechanical test methods that measure the stress index mechanically, etc. In other words, analyzing human psychology may be possible only after psychological analysis experts observe and counsel a recipient for a long time, but after a long study of psychologists, it was found that the objective and uniform methods described above may obtain satisfactory results to only some extent.

In addition, in order to obtain a more accurate psychological diagnosis result with respect to a diagnosis recipient, a diagnostician does not use only one analysis method, but uses several analysis methods simultaneously or sequentially. That is, the diagnostician diagnoses psychology of the diagnosis recipient by using a plurality of psychological diagnosis results obtained through a plurality of analysis methods.

However, when various analysis methods are performed on one diagnosis recipient simultaneously or sequentially, there is a problem in that a lot of time and manpower are required to obtain a single psychological diagnosis result.

In addition, there are not many competent experts, and there is a problem in that a lot of physical time and money are incurred to receive psychological counseling from competent experts.

### DESCRIPTION OF EMBODIMENTS

### Technical Problem

The present disclosure provides an electronic device for performing psychological counseling through pictures.

The present disclosure also provides extracting detailed attribute information about a drawing process of a counseling recipient, and determining a psychological state of the counseling recipient in consideration of the detailed attribute information obtained during the drawing process in addition to a final image obtained through drawing.

The present disclosure also provides transferring a final image, which is input data representing a psychological state of a counseling recipient, and detailed attribute information obtained during a drawing process, to a psychological analysis expert group and obtaining detailed analysis comments.

### Solution to Problem

According to the embodiments of the present disclosure, a method of determining a psychological state of a counseling recipient through a drawing process includes receiving, by a user terminal, a psychological test start input with respect to the counseling recipient; setting an area to be drawn in by the counseling recipient, in response to the psychological test start input; outputting, by the user terminal, one or more pieces of instruction information in an order of registration, storing the area to be drawn in at a certain frame rate for a certain period of time from a time at which the instruction information is output, and obtaining drawing data corresponding to the one or more pieces of instruction information;
receiving, by the user terminal, a psychological test end input and sensing a final image in the area to be drawn in; and transmitting, by the user terminal, the drawing data and the final image to a management server, and receiving analysis comments from a psychological counseling expert who has analyzed the drawing data and the final image from the management server.

The method may further include selecting, by the management server receiving the drawing data and the final image, the drawing data and the final image, and selecting a psychological counseling expert group corresponding to the counseling recipient; and transmitting a request for analysis of the drawing data and the final image to one or more electronic devices included in the psychological counseling expert group.

The method may further include receiving, by the user terminal, analysis comments from the management server, and outputting, through an output unit, detailed information comprising the analysis comments of the drawing data for each time and the number of received analysis comments which are included in an icon expressing the drawing data through an output unit.

The method may further include receiving, by the user terminal, analysis comments from the management server, and classifying the analysis comments with respect to one or more objects included in the final image, and generating and providing information hierarchized in consideration of a relationship between the objects.

A computer program according to an embodiment of the present disclosure may be stored in a medium to execute a method of determining a psychological state through a drawing process of a counseling recipient according to an embodiment of the present disclosure by using a computer.

Moreover, a computer-readable recording medium having recorded thereon a computer program for executing other methods, other systems, and the method for implementing the present disclosure is further provided.

In addition to the aforesaid details, other aspects, features, and advantages will be clarified from the following drawings, claims, and detailed description.

### Advantageous Effects of Disclosure

According to an embodiment of the present disclosure, an electronic device for performing psychological counseling through pictures may be provided.

According to an embodiment of the present disclosure, an electronic device for extracting detailed attribute information about a drawing process made by a counseling recipient, and determining a psychological state of the counseling recipient in consideration of the detailed attribute information obtained during the drawing process in addition to a final image obtained through drawing may be provided.

According to an embodiment of the present disclosure, an electronic device for transferring a final image which is input data representing a psychological state of a counseling recipient, and detailed attribute information obtained during a drawing process to a psychological analysis expert group and obtaining detailed analysis comments may be provided.

### BRIEF DESCRIPTION OF DRAWINGS

FIG. 1 is a block diagram of a psychological counseling program for determining a psychological state of a counseling recipient by obtaining a drawing process of the counseling recipient, according to embodiments of the present disclosure.

FIG. 2 is a block diagram of a user terminal according to embodiments of the present disclosure.

FIGS. 3 to 8 are flowcharts of a psychological counseling method according to embodiments of the present disclosure.

FIG. 9 is a diagram of a network environment including a user terminal, management servers, etc., according to embodiments of the present disclosure.

FIG. 10 is an example diagram of a user interface displaying analysis comments on a drawing.

FIG. 11 is an example diagram of a user interface inputting analysis comments on drawing data in a terminal of an expert.

FIG. 12 is a diagram illustrating a network environment according to embodiments of the present disclosure.

### BEST MODE

As the present disclosure allows for various changes and numerous embodiments, particular embodiments will be illustrated in the drawings and described in detail in the written description. The effects and features of the present disclosure, and ways to achieve them will become apparent by referring to embodiments that will be described later in detail with reference to the drawings. However, the present disclosure is not limited to the following embodiments but may be embodied in various forms.

Expressions such as “include” or “may include” that may be used in various embodiments of the present disclosure indicate the existence of a disclosed corresponding function, operation, or component, and do not limit one or more additional functions, operations, or components, etc. Also, in various embodiments of the present disclosure, it should be understood that the terms “include” or “have” specify the presence of stated features, integers, steps, operations, components, parts or combinations thereof, but do not preclude possibility of the presence or addition of one or more other features, integers, steps, operations, components, parts or combinations thereof.

In various embodiments of the present disclosure, expressions such as “or” include any and all combinations of words listed together. For example, “A or B” may include A, may include B, or may include both A and B.

In various embodiments of the present disclosure, although the terms first, second, etc. may be used to describe various components of various embodiments, these embodiments should not be limited by these terms. For example, the above expressions do not limit the order and/or importance of corresponding components. The above expressions may be used to distinguish one component from another. For example, both a first user device and a second user device are user devices, and represent different user devices. For example, without departing from the scope of the various embodiments of the present disclosure, a first component may be referred to as a second component, and similarly, a second component may also be referred to as a first component.

When a component is referred to as being “connected” or “coupled” to another component, the component may be directly connected to or coupled to the other component, but it should be understood that another new component may exist between the component and the other component. When a component is referred to as being “directly connected” or “directly coupled” to another component, the component may be directly connected to or coupled to the other component, but it should be understood that another new component does not exist between the component and the other component.

In an embodiment of the present disclosure, the terms such as “module”, “unit”, “part”, etc. are terms for designating a component that performs at least one function or operation, and such component may be implemented as hardware or software or implemented as a combination of hardware and software. In addition, a plurality of “modules”, “units”, “parts”, etc. may be integrated into at least one module or chip and may be implemented as at least one processor, except when each needs to be implemented as individual specific hardware.

The terminology used in various embodiments of the present disclosure is only used to describe one specific embodiment, and is not intended to limit the various embodiments of the present disclosure. The singular forms “a”, “an”, and “the” are intended to include the plural forms as well, unless the context clearly indicates otherwise.

Unless otherwise defined, all terms including technical and scientific terms used herein have the same meaning as commonly understood by one of ordinary skill in the art to which various embodiments of the present disclosure belong.

It should be understood that terms, such as those defined in commonly used dictionaries, should be interpreted as having a meaning that is consistent with their meaning in the context of the relevant art and should not be interpreted in an idealized or overly formal sense unless expressly so defined herein.

Hereinafter, various embodiments of the present disclosure will be described in detail with reference to the accompanying drawings.

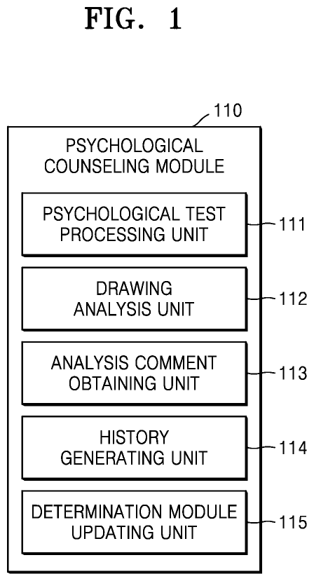

FIG. 1 is a block diagram of a psychological counseling program 110 for determining a psychological state of a counseling recipient by obtaining a drawing process of the counseling recipient according to embodiments of the present disclosure.

The psychological counseling module 110 may obtain the drawing process of the counseling recipient, collect analysis comments that has analyzed the psychological state of the counseling recipient through attribute information of the drawing process and a final image obtained in the drawing process, and based on this, provide the overall psychological state. The psychological counseling module 110 may transmit the obtained drawing data to a management server, provide the drawing process of the counseling recipient and the final image to a plurality of analysis experts through the management server, and receive not only the overall analysis comment but also an analysis comment on the drawing process.

The psychological counseling module 110 may receive the analysis comments from a psychological counseling expert through the management server without directly meeting the psychological counseling expert. The psychological counseling module 110 may be implemented to perform psychological counseling through not only the final image drawn in for psychological counseling, but also images storing the drawing process as digital data. The psychological counseling module 110 may periodically update a determination model trained through the psychological counseling result data made by psychological counseling experts from the management server, and determine the psychological state of the counseling recipient through the determination model.

The psychological counseling module 110 may be received from an external server over a communication network and installed. The psychological counseling module 110 to which a new function is periodically added may be received and installed. The psychological counseling module 110 may be stored in a storage medium or the like and provided in a user terminal 100. In addition, the psychological counseling module 110 may be stored in a separate storage medium and electrically connected to the user terminal 100 to operate by one or more processors of the user terminal 100.

The psychological counseling module 110 may include a psychological test processing unit 111, a drawing analysis unit 112, an analysis comment obtaining unit 113, a history generating unit 114, and a determination module updating unit 115.

The psychological test processing unit 111 may start a psychological test through an input of the counseling recipient. The psychological test processing unit 111 may set a range of the final drawing image by the counseling recipient in response to an input for starting the psychological test.

The psychological test processing unit 111 outputs drawing instruction information, and obtains frames in which the drawing process of the counseling recipient corresponding to the drawing instruction information is stored as digital data. Here, the drawing instruction information may be stored in advance or received from the management server. Also, the drawing instruction information may be output through another user terminal. That is, the drawing instruction information may be output through a first user terminal, and the drawing process corresponding to the drawing instruction information may be input through a second user terminal. At this time, the first user terminal may display first drawing instruction information through the output unit, and transmit an output signal of a drawing area corresponding to the displayed first drawing instruction information to the second user terminal. The second user terminal may obtain the drawing process and data on the drawing process through the corresponding drawing area, and transmit data on the drawing process to the first user terminal. The first user terminal may perform a psychological test while sequentially outputting a plurality of drawing instruction information for the psychological test of a user. The first and second user terminals may interwork to perform psychological counseling.

Here, the drawing process is stored as digital data at a previously set frame rate. The frame rate may be set by each user or set by an expert. In addition, the frame rate may vary according to output instruction information. Drawing data with respect to first instruction information may be stored at a first frame rate, and drawing data with respect to second instruction information may be stored at a second frame rate.

The psychological test processing unit 111 may obtain drawing data acquired during the drawing process in time series corresponding to the drawing instruction information and obtain a final drawing image after the drawing process is completed. The psychological test processing unit 111 may obtain the drawing data and the final drawing image for each drawing instruction information. In this regard, before obtaining the final drawing image, the psychological test processing unit 111 may receive an end signal of the drawing process corresponding to the corresponding drawing instruction information from the user. The psychological test processing unit 111 may obtain the drawing data stored at the frame rate and the final drawing image. Here, the frame rate may be set differently according to setting information included in each instruction information, but is not limited thereto, and may be fixed to one value in one drawing process.

The psychological test processing unit 111 may generate an expert group based on personal information of the counseling recipient and/or the final drawing image. Information about the expert group may be received from the management server and generated. The psychological test processing unit 111 may request expert group information corresponding to the counseling recipient from the management server and generate the expert group in correspondence to a response to a request.

The psychological test processing unit 111 may request an analysis comment on the final drawing image with an account of the expert group, and obtain an analysis comment on the drawing data. Here, the drawing data may include images captured in correspondence to the drawing instruction information and the final image generated during the drawing process.

The psychological test processing unit 111 may obtain the drawing process and the final drawing image by using a provided drawing tool. The drawing process and the final drawing image may be converted into digital data and stored. The psychological test processing unit 111 may classify analysis comments on the final drawing image and/or the images (a frame set) storing the drawing process as the digital data in correspondence to each of the drawing order, location and arrangement information of lower objects included in an object, location and arrangement information of objects, drawing order information of the lower objects included in the object, line thickness information, color distribution information, etc. More specifically, the analysis comment may be added with respect to the overall drawing process and the line thickness information and the color distribution information of the drawing in the final image, but is not limited thereto. The analysis comment may be individually added with respect to the drawing order, objects expressed by drawing, detailed lower objects included in the objects, a relative expression method between the objects, a relative expression method between the lower objects, etc. The psychological test processing unit 111 may extract data with respect to the drawing based on analysis data analyzed through the drawing analysis unit 112.

Through this, a device capable of determining a psychological state by further considering not only the final drawing image by the counseling recipient but also time series information about the drawing process, relative information reflecting art ability of a user, etc. may be provided.

The drawing analysis unit 112 may analyze the drawing data to extract the line thickness information and the color distribution information of the drawing process and the final drawing image. The drawing data and/or the line thickness information of the final drawing image may include accumulated time information for each line thickness. The drawing analysis unit 112 may analyze the drawing data to count times drawn in with a first line thickness and calculate accumulated time information with respect to the first line thickness. The line thickness of the drawing may be set to one of a plurality of previously set options. The color distribution information may include a distribution ratio for each color based on information about all pixels in the final drawing image. The drawing analysis unit 112 may extract color information of pixels and generate the color distribution information based on the number of pixels for each of the color information. The drawing analysis unit 112 may extract the drawing order of objects in consideration of the frames in which the drawing process is stored as the digital data. In detail, the drawing analysis unit 112 may extract an object for each frame and then make an object included in the frame correspond to each of frames arranged in chronological order. The drawing analysis unit 112 may classify frames corresponding to one object into one bundle, and extract drawing orders between objects based on the drawing order of bundles of the frames with respect to each object. For example, the drawing analysis unit 112 may classify first frames of a first object and second frames of a second object, and determine a drawing order of the objects in consideration of the order of the first frames and the second frames.

The drawing analysis unit 112 may analyze the final image received from the psychological test processing unit 111 to extract location, size, and arrangement information of the objects.

The drawing analysis unit 112 may extract a drawing order of the lower objects within the object. The drawing analysis unit 112 may analyze a frame set with respect to the first object to extract a drawing order of lower objects included in the first object. The drawing analysis unit 112 extracts the lower objects of the first object from the final image. The drawing analysis unit 112 may extract the lower object of the first object corresponding to the frame and then make the lower object correspond to each of the frames arranged in chronological order, classify the frames corresponding to one lower object into one bundle, and extract drawing orders of the lower objects of the first object based on the drawing order of bundles of the frames with respect to each lower object. The drawing analysis unit 112 may extract the lower objects included in each of the frames corresponding to the first object, and classify the frames corresponding to the first object according to the extracted lower objects, thereby extracting the drawing order of the lower objects of the first object.

The drawing analysis unit 112 may analyze the drawing data to extract location values, size values, and arrangement information of lower objects included in one object. A user terminal may analyze the final image to extract location values, size values, and arrangement information of one or more lower objects included in the first object.

The drawing analysis unit 112 may extract relative information reflecting the art ability of the user. The drawing analysis unit 112 may compare a specific expression degree of the objects with a subjective art ability of each user to calculate an object expression index which is subjectively assessed for each object. Here, the object expression index, which is an index set by comprehensively measuring to what extent each object has been specifically expressed, a drawing time distribution ratio with another object included in one drawing, etc., may be determined for each object included in the drawing generated by each user based on art ability information of each user. At this time, to what extent each object has been specifically expressed may be determined by comparing the number of lower objects included in each object, the drawing time of the lower objects, the number of colors included in expressing the lower objects, etc. with the art ability information of each user usually expressing the corresponding object. The distribution ratio of the drawing time refers to a distribution ratio of drawing times of each of the lower objects in the total drawing time of the object.

The analysis comment obtaining unit 113 may receive analysis comments on the drawing process and the line thickness information and/or the color distribution information with respect to the final drawing image. The analysis comment obtaining unit 113 may automatically translate and provide the analysis comments into a language of each user by using an automatic translation module.

The analysis comment obtaining unit 113 may determine the psychological state of the recipient based on the drawing process and the line thickness information and the color distribution information with respect to the final drawing image obtained through the drawing analysis unit 112. The analysis comment obtaining unit 113 may calculate the accumulated time for each line thickness based on the drawing process and the final drawing image, and determine the psychological state of the recipient in correspondence to the accumulated time for each line thickness. The analysis comment obtaining unit 113 may extract color information of pixels of the final drawing image, count the number of pixels for each color, and determine the psychological state of the recipient based on the number of pixels for each color, a distribution ratio, etc.

The analysis comment obtaining unit 113 may analyze the analysis comments on the drawing for psychological counseling to generate a comment history. The comment history may include analysis comments received from experts on the drawings and the analysis comments may include a time-series representation. The comment history may be in the form of an image in which a process of generating the analysis comments is reproduced in time series. The comment history may be provided in the form of being connected to objects and/or lower objects included in the drawing corresponding to the analysis comments, and may be sequentially expressed according to the order in which each analysis comment is received from the expert.

The analysis comment obtaining unit 113 may analyze the analysis comments on the drawing and receive, from the management server, a result of searching for a similar counseling recipient group having a similar drawing pattern and/or a similar analysis comment to the counseling recipient. The analysis comment obtaining unit 113 may transmit a search input for the counseling recipient group with respect to the analysis comment on the drawing to the management server. The analysis comment obtaining unit 113 may provide information about the similar counseling recipient group received from the management server through an output unit. In this regard, the management server may provide the user terminal 100 with personal information and/or detailed psychological state which are/is hidden processed in the information about the similar counseling recipient group.

The analysis comment obtaining unit 113 may process a paid payment on the psychological state information with respect to the similar counseling recipient group and then provide the information about the similar counseling recipient group that is received again from the management server and is not hidden processed, through the output unit.

The analysis comment obtaining unit 113 may automatically generate analysis comments on information (the frames in which the drawing process is stored as the digital data, the location, size and arrangement information of the objects, the drawing order of the lower objects within the object, the object expression index reflecting the art ability of the user, etc.) obtained by using algorithm generated by linking the analysis comments generated by an actual expert group with detailed items (the objects, the lower objects, the drawing order, the drawing time series information, the object expression index, etc.) included in the drawing process.

The analysis comment obtaining unit 113 may provide a user interface for requesting payment processing on a payment amount determined based on the number of the obtained analysis comments and rating information. The payment amount may be determined through an external management server. The analysis comment obtaining unit 113 may receive a payment processing interface with respect to the payment amount determined based on the number of analysis comments and rating information from the external management server. In addition, the management server may accumulate a certain portion of the amount paid through the counseling recipient as the ID of the diagnostician of each analysis comment.

The analysis comment obtaining unit 113 may provide a function of receiving assessment information about the obtained analysis comment.

The analysis comment obtaining unit 113 may arrange and provide the analysis comments that are accumulatively obtained in a chronological order. For example, the analysis comment obtaining unit 113 may generate and provide history information related to an analysis comment input time, such as 10 pieces at a first time point, 8 pieces at a second time point, etc. after the drawing. The analysis comment obtaining unit 113 may make the analysis comment correspond to each detailed object included in the drawing data and provide the analysis comment. In addition, the analysis comment is provided with detailed information, and the analysis comment obtaining unit 113 may provide the analysis comment including rating of the expert who is a creator, the experience of the expert, and the rating for the analysis comment.

The community function providing unit 114 may provide one or more users included in the similar counseling recipient group and a community function included in one group. The community function providing unit 114 may provide a chat room connected to terminals of the one or more users whose information is publicly processed through a paid payment. The community function providing unit 114 may add an expert to the corresponding chat room upon a request from a user and process message transmission/reception for group diagnosis from the counselor. Specifically, the community function providing unit 114 may generate one chat room, send an invitation message to the terminals of the one or more users included in the similar counseling recipient group and/or a terminal of the expert in the corresponding chat room, and send and receive messages with users who have entered the chat room through the invitation message. The corresponding chat room may be implemented so as to exchange messages anonymously without disclosing personal information, and implemented so as not to express any indication even with respect to the expert.

The determination module update unit 115 may receive analysis comments on the counseling recipient received from a plurality of experts, from the management server. The determination module update unit 115 may classify the analysis comments with respect to the creator (the expert). The determination module update unit 115 may classify the analysis comments with respect to objects and lower objects, and may hierarchize the analysis comments in consideration of relationships between the objects. For example, when a drawing including a first object, a second object, and a third object is generated by the counseling recipient, the determination module update unit 115 may classify analysis comments on the first object, analysis comments on the second object, and analysis comments on the third object, and classify and provide the analysis comments on the first object for each lower object of the first object. The determination module update unit 115 may provide an analysis comment of a 1-1th lower object of the first object and an analysis comment of a 1-2th lower object of the first object differently. The lower object refers to detailed elements included in the object and/or the drawing order, the arrangement information, etc. For example, when the object is a face, eyes, nose, mouth, eyebrows, and ears may be the lower objects. In addition, the order of drawing eyes, nose, mouth, eyebrows, ears, etc., relative size and/or location information between eyes and nose may also be the lower objects.

The management server receiving the data from the determination module update unit 115 may generate a determination module that determines a psychological state from the drawing data by using the hierarchized analysis comments and/or the drawing data received from one or more user terminals. It may be verified as to whether the generated determination module has reliability equal to or greater than a certain level. The verified determination module may be transmitted to the user terminal and update the psychological counseling module 110.

The psychological counseling module 110 may further include a component for the expert group in addition to the component for a psychology recipient. The psychological counseling module 110 may further include an analysis comment input unit (not shown) provided to the expert group.

The analysis comment input unit may provide a user interface for inputting an analysis comment through the output unit when a status of a logged-in user is determined to be the expert group. The analysis comment input unit may control to provide drawing data corresponding to the corresponding expert, among drawing data in which personal information of the psychology recipient is not disclosed, and to input analysis comments on the drawing data. The analysis comment may be controlled to be input for each object of the drawing data. For example, the analysis comment may be input with respect to the detailed items extracted from the drawing data, such as each object, the drawing order, line thickness, color distribution, etc.

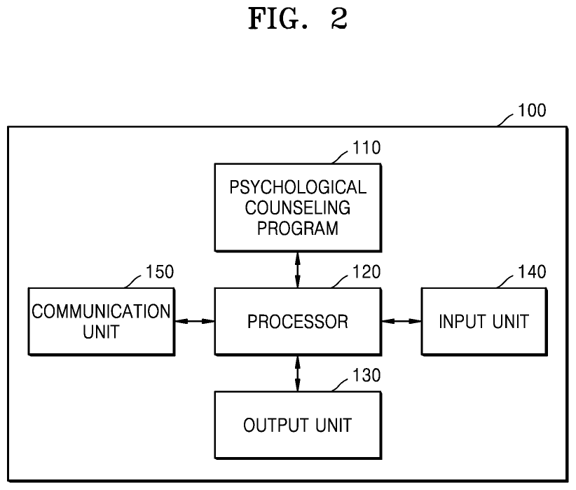

FIG. 2 is a block diagram of the user terminal 100 according to embodiments of the present invention.

As shown in FIG. 2, the user terminal 100 may include the psychological counseling module 110, a processor 120, an output unit 130, an input unit 140, and a communication unit 150.

The processor 120 is a configuration for generally controlling the user terminal 100. Specifically, the processor 120 controls the general operation of the user terminal 100 by using various programs stored in the psychological counseling module 110 of the user terminal 100. For example, the processor 120 may include a central processing unit (CPU), a RAM, a ROM, and a system bus. Here, the ROM is a configuration in which an instructions set for system booting is stored, and the CPU copies an operating system (OS) stored in a memory of the user terminal 100 to the RAM according to instructions stored in the ROM and executes the OS to boot the system. When booting is completed, the CPU may copy various applications stored in the psychological counseling module 110 to the RAM and execute the applications to perform various operations. Although it has been described above that the user terminal 100 includes only one CPU, the user terminal 100 may be implemented as a plurality of CPUs (or digital signal processors (DSPs), system-on-chips (SoCs), etc.)

According to an embodiment of the present disclosure, the processor 120 may be implemented as a DSP, a microprocessor, or a time controller (TCON) that processes a digital signal. However, the processor 120 is not limited thereto, and the processor 120 may include one or more of a CPU, a micro controller unit (MCU), a micro processing unit (MPU), a controller, an application processor (AP), a communication processor (CP) or an advanced reduced instruction set computing (RISC) machine (ARM) processor, or may be defined as the corresponding term. In addition, the processor 120 may be implemented by a SoC or a large scale integration (LSI) in which a processing algorithm is embedded, or may be implemented in a field programmable gate array (FPGA) form.

The output unit 130 may display a drawing process, a final drawing image, and analysis comments on the drawing generated by the user terminal 100 by using the psychological counseling module 110. According to an embodiment of the present disclosure, the output unit 130 may display a user interface according to a user input that is input through the input unit 140. The output unit 130 may output graphic data, visual data, auditory data, vibration data, and pressure data that are stored under the control of the psychological counseling module 110.

The output unit 130 may be implemented as various types of display panels. For example, the display panel may be implemented in various types of displays such as a liquid crystal display (LCD), organic light emitting diode (OLED), active-matrix organic light-emitting diode (AM-OLED), liquid crystal on silicon (LcoS), digital light processing (DLP), etc. Also, the output unit 130 may be combined with at least one of a front region, a side region, and a rear region of the display panel in the form of a flexible display.

The output unit 130 may be implemented as a touch screen having a layer structure. The touch screen may have a function of detecting not only a display function but also a touch input location, a touched area, and even a touch input pressure, and may also have a function of detecting not only a real-touch but also a proximity touch.

The input unit 140 may include a user interface for inputting various information into the user terminal 100.

The communication unit 150 is a configuration for transmitting/receiving data to and from devices such as a server and other user terminals. The communication unit 150 may include a near field communication unit such as a Bluetooth communication unit, a Bluetooth low energy (BLE) communication unit, a near field communication unit, a WLAN (Wi-Fi) communication unit, a Zigbee communication unit, an infrared data association (IrDA) communication unit, a Wi-Fi direct (WFD) communication unit, an ultra wideband (UWB) communication unit, and an Ant+ communication unit, and a mobile communication network.

The user terminal 100 may further include a storage medium (not shown) storing various data for overall operations of the processor 120, such as a program for processing or controlling of the processor 120. The storage medium may store a plurality of application programs (or applications) driven in the user terminal 100, data for an operation of the user terminal 100, and instructions. At least some of such application programs may be downloaded from an external server through wireless communication. In addition, at least some of the application programs may be present on the user terminal 100 from the time of shipment for a basic function of the user terminal 100. The application program may be stored in the storage medium, and may be driven by the processor 120 to perform the operation (or the function) of the user terminal 100.

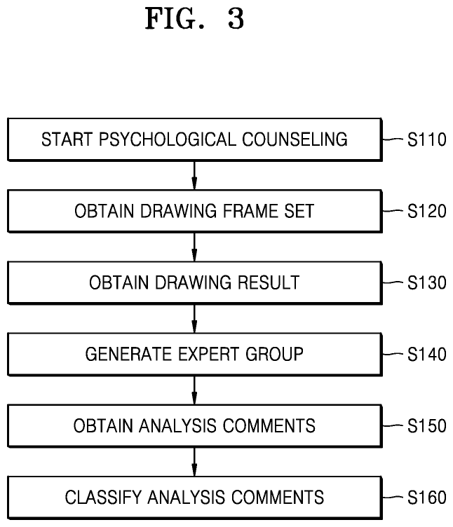

FIGS. 3 to 8 are flowcharts of a psychological counseling method according to embodiments of the present disclosure.

As shown in FIG. 3, in S110, the user terminal 100 may start a psychological test through an input of a counseling recipient. The user terminal 100 may set the range of a final drawing image by the counseling recipient in response to an input for starting the psychological test.

In S120, the user terminal 100 outputs drawing instruction information and obtains frames in which a drawing process of the counseling recipient corresponding to the drawing instruction information is stored as digital data.

Here, frames in which the drawing process is stored as the digital data may be stored at a previously set frame rate.

In S130, the user terminal 100 may obtain the final drawing image through drawing.

In S140, the user terminal 100 may generate an expert group based on the counseling recipient and/or the final drawing image.

In S150, the user terminal 100 may request an analysis comment on the final drawing image with as account of the expert group and obtain the analysis comment.

In S160, the user terminal 100 may classify analysis comments on the final drawing image and/or a frame set storing the drawing process as the digital data in correspondence to each of a drawing order, location and arrangement information of lower objects included in an object, location and arrangement information of objects, drawing order information of the lower objects included in the object, etc.

Through this, a device capable of determining a psychological state by further considering not only the final drawing image by the counseling recipient but also time series information about the drawing process may be provided.

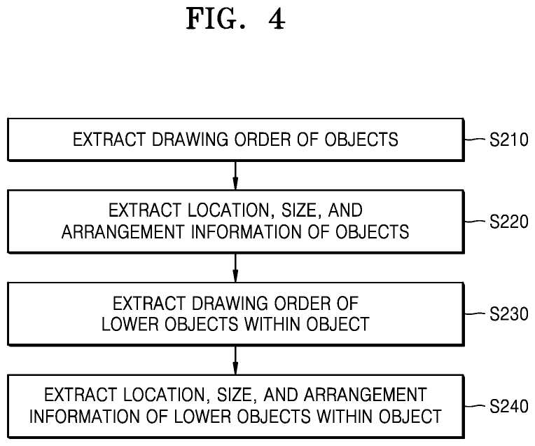

As shown in FIG. 4, in S210, the user terminal 100 may extract the drawing order of objects in consideration of the frames in which the drawing process is stored as the digital data. Here, the object refers to objects, people, flowers, sky, house, tree, fog, cloud, etc. included in the drawing. The drawing order of objects refers to the drawing order by the user. Specifically, the user terminal 100 may extract an object for each frame and then make an object included in the frame correspond to each of frames arranged in chronological order, thereby extracting the drawing orders of the objects.

In S220, the user terminal 100 may analyze the final image to extract location, size, and arrangement information of the objects.

In S230, the user terminal 100 may extract a drawing order of the lower objects within the object. The user terminal 100 may analyze a frame set with respect to a first object to extract a drawing order of lower objects included in the first object.

The user terminal 100 extracts the lower objects of the first object from the final image. The user terminal 100 may extract the lower object of the first object with respect to each frame and then make the lower object correspond to each of the frames arranged in chronological order, and extract drawing orders of the lower objects of the first object in consideration of the order of the frames.

In S240, the user terminal 100 may extract location, size, and arrangement information of the lower object within the object. The user terminal 100 may analyze the final image to extract location, size, and arrangement information of one or more lower objects included in the first object.

Through this, the user terminal 100 may extract the drawing order of the objects, the location, size, and arrangement information of the objects, the drawing order of the lower objects, the location, size, and arrangement information of the in an upper object.

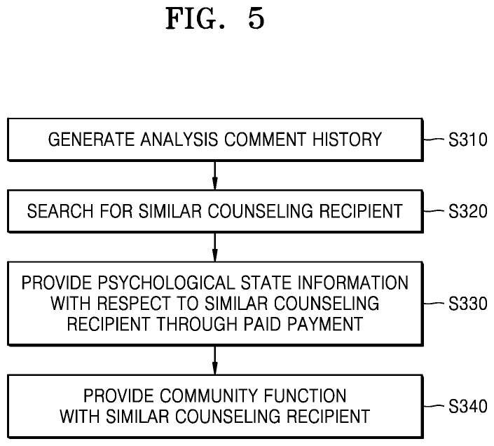

As shown in FIG. 5, in S310, the user terminal 100 may analyze the analysis comments on the drawing for psychological counseling to generate a comment history. The comment history, which is data in which analysis comments received from experts with respect to drawing are expressed in time series, may be in the form of an image in which a process of generating the analysis comments is reproduced in time series. The comment history may be provided in the form of being connected to objects and/or lower objects included in the drawing corresponding to the analysis comments, and may be sequentially expressed according to the order in which each analysis comment is received from the expert.

In S320, the user terminal 100 may analyze the analysis comments on the drawing and receive, from the management server, a result of searching for a similar counseling recipient group having a similar drawing pattern and/or a similar analysis comment to the counseling recipient. The user terminal 100 may transmit a search input for the counseling recipient group with respect to the analysis comment on the drawing to the management server. The user terminal 100 may provide information about the similar counseling recipient group received from the management server through an output unit. The management server may provide the user terminal 100 with personal information and/or detailed psychological state which are/is hidden processed in the information about the similar counseling recipient group.

In S330, the user terminal 100 may process a paid payment on the psychological state information with respect to the similar counseling recipient group and then provide the information about the similar counseling recipient group that is received again from the management server and is not hidden processed, through the output unit.

In S340, the user terminal 100 may provide one or more users included in the similar counseling recipient group and a community function included in one group. The user terminal 100 may provide a chat room connected to terminals of the one or more users whose information is publicly processed through a paid payment. The user terminal 100 may add an expert to the corresponding chat room upon a request from a user and process message transmission/reception for group diagnosis from the counselor. Specifically, the user terminal 100 may generate one chat room, send an invitation message to the terminals of the one or more users included in the similar counseling recipient group and/or a terminal of the expert in the corresponding chat room, and send and receive messages with users who have entered the chat room through the invitation message. The corresponding chat room may be implemented so as to exchange messages anonymously without disclosing personal information, and implemented so as not to express any indication even with respect to the expert.

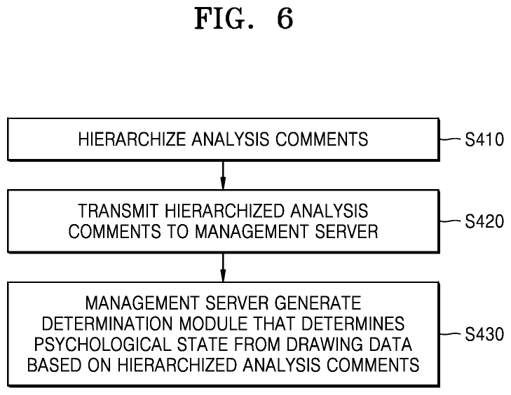

As shown in FIG. 6, in S410, the user terminal 100 may receive analysis comments on the counseling recipient received from a plurality of experts, from the management server. The user terminal 100 may classify the analysis comments with respect to a creator (an expert). The user terminal 100 may classify the analysis comments with respect to objects and lower objects, and may hierarchize the analysis comments in consideration of relationships between the objects. when a drawing including a first object, a second object, and a third object is generated by the counseling recipient, the user terminal 100 may classify analysis comments on the first object, analysis comments on the second object, and analysis comments on the third object, and classify and provide the analysis comments on the first object for each lower object of the first object. The user terminal 100 may provide an analysis comment of a 1-1th lower object of the first object and an analysis comment of a 1-2th lower object of the first object differently. The lower object refers to detailed elements included in the object and/or the drawing order, the arrangement information, etc. For example, when the object is a face, eyes, nose, mouth, eyebrows, and ears may be the lower objects. In addition, the order of drawing eyes, nose, mouth, eyebrows, ears, etc., relative size and/or location information between eyes and nose may also be the lower objects.

In S430, the management server may generate a determination module that determines a psychological state from the drawing data by using the hierarchized analysis comments and/or the drawing data received from one or more user terminals. It may be verified as to whether the generated determination module has reliability equal to or greater than a certain level. The verified determination module may be transmitted to the user terminal and update the psychological counseling program.

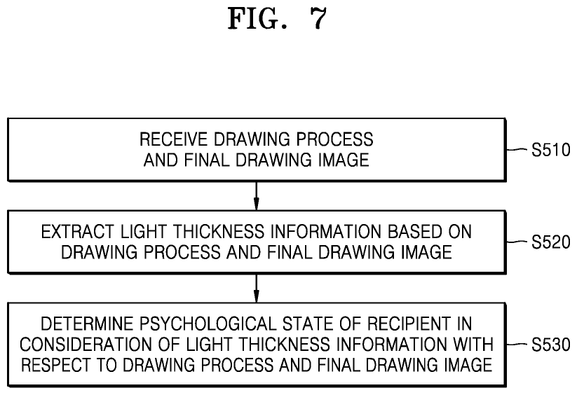

As shown in FIG. 7, in S510, the user terminal 100 may obtain the drawing process and the final drawing image through drawing. The user terminal 100 may obtain the drawing process by using a previously set drawing tool, and finally obtain the final drawing image.

In S520, the user terminal 100 may extract line thickness information based on the drawing process and the final drawing image. The user terminal 100 may draw with the first line thickness for the first time and may draw with the second line thickness for the second time. The user terminal 100 may extract the first time drawn with first line thickness and the second time drawn with the second line thickness through the drawing process. That is, the user terminal 100 may extract accumulated time information for each line thickness of the drawing touch during the drawing process. The user may complete the drawing by using 2 or more line thicknesses.

In S530, the user terminal 100 may collect psychological states of the recipient corresponding to the line thickness information including the accumulated time information for each line thickness of the drawing touch and determine the current psychological state of the recipient.

In S610, the user terminal 100 may obtain the drawing process and the final drawing image through drawing. The user terminal 100 may obtain the drawing process by using the previously set drawing tool, and obtain the final drawing image.

In S620, the user terminal 100 may extract color distribution information based on the drawing process and the final drawing image. The user terminal 100 may draw with a first color of a first number of pixels and may draw with a second color of a second number of pixels. The user terminal 100 may calculate a distribution ratio of the first number of pixels expressed in the first color, a distribution ratio of the second number of pixels expressed in the second color and the like, among the total number of pixels of the final drawing image.

In S630, the user terminal 100 may collect psychological states of the recipient corresponding to the color distribution information including the distribution ratio for each color and determine the current psychological state of the recipient.

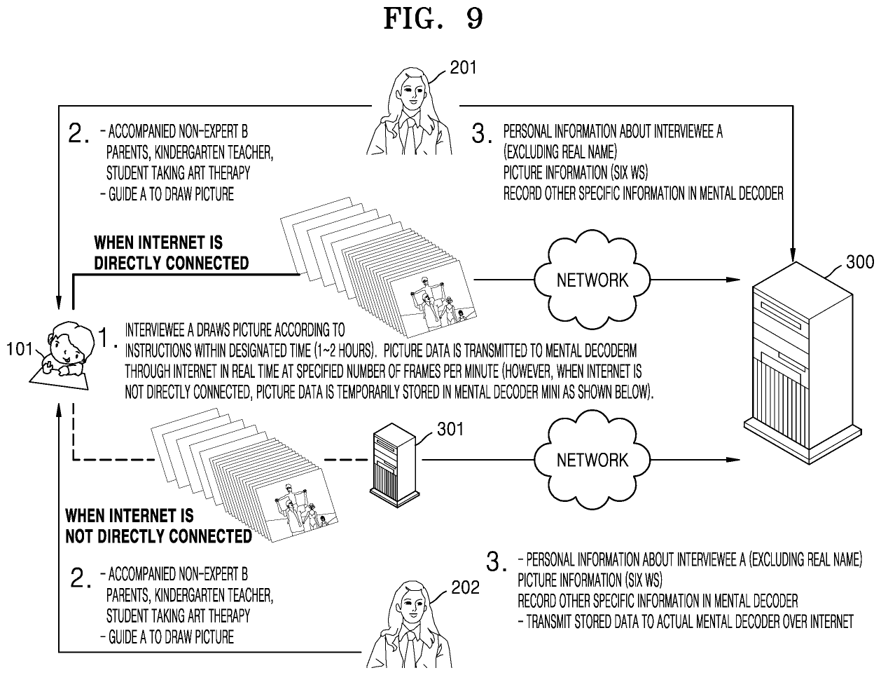

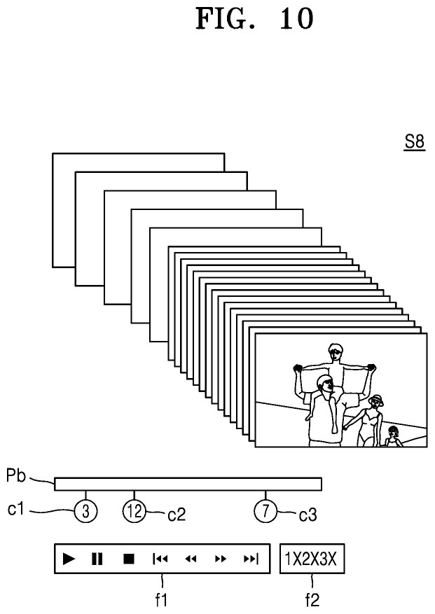

FIG. 9 is a diagram of a network environment including the user terminal 100, management servers 300 and 301, etc. according to embodiments of the present invention.

As shown in FIG. 9, a counseling recipient 101 may perform drawing for assessment of a psychological state through the user terminal 100. The performed drawing process and a final drawing image may be input through the user terminal 100.

A psychological state assessment process of the counseling recipient 101 may be performed through a terminal of a user 201 accompanied with the counseling recipient 101. Alternatively, the drawing data may be obtained through a terminal of the counseling recipient 101, and drawing instruction data may be obtained through the terminal of the user 201 accompanied with the counseling recipient 101.

Psychological counseling start and end signals may be obtained through the terminal of the user 201 accompanied with the counseling recipient 101.

The management servers 300 and 301 may operate according to a network connection state. When a network is connected, the user terminal 100 may be connected to the management server 300 that communicates remotely, and when the network is not connected, the user terminal 100 may be connected to the management server 301 connected locally so that a psychological counseling service may be executed.

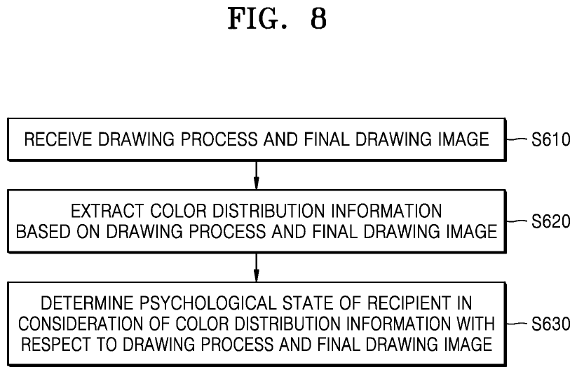

The user terminal 100 may provide a user interface S8 displaying analysis comments on the drawing as shown in FIG. 8.

The user interface S8 may provide a progress bar Pb of the drawing data, an icon indicating number information c1, c2, and c3 of analysis comments of time point of the drawing data, a reproduction-related function f1, and an output speed function f2.

c1 of the drawing data denotes that 3 analysis comments were collected at a first time point. c2 of the drawing data may denote that 12 analysis comments were collected at a second time point.

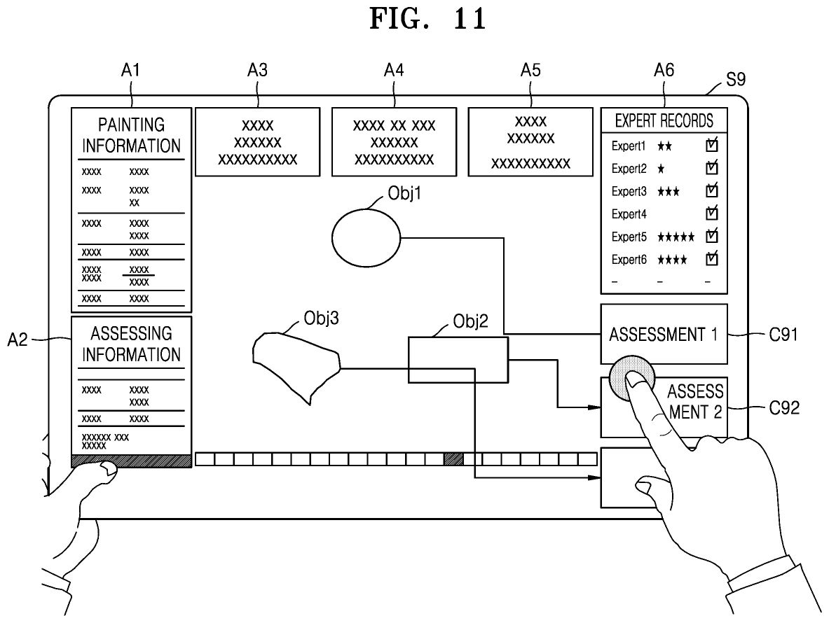

FIG. 11 is an example diagram of a user interface S9 inputting analysis comments on drawing data in a terminal of an expert.

The user interface S9 inputting the analysis comments may make an analysis comment correspond to each object in the drawing data and input the comments. The user interface S9 may input analysis comments Assessment 1, Assessment 2, and Assessment 3 through a closed curve including objects obj1, obj2, and obj3 and areas inputting the objects obj1, obj2, and obj3 into a lower left end.

The user interface S9 may include an area A1 providing information about drawing, an area A2 providing information about analysis comments, and areas A3, A4, and A5 providing information about objects.

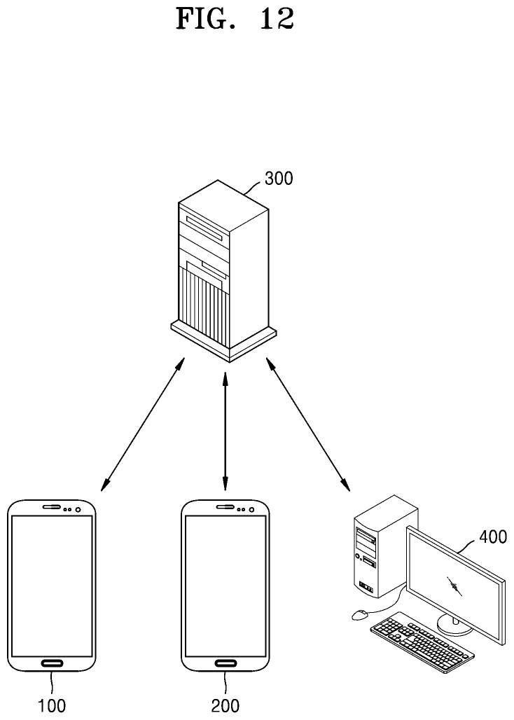

FIG. 12 is a diagram illustrating a network environment according to embodiments of the present disclosure.

A psychological counseling system may include the terminal 100 of a counseling recipient, the terminal 200 of a user accompanied with the counseling recipient, the management server 300, and a terminal 400 of an expert.

The terminal 100 of the counseling recipient may provide a psychological counseling service. In FIG. 12, the terminal 100 of the counseling recipient is expressed in the singular, but the number is not limited. The counseling recipient may capture or store a drawing process as images through the terminal 100. In this case, a psychological counseling start signal may be further received through the terminal 200 of the user accompanied with the counseling recipient. That is, the management server 300 may receive a psychological counseling start signal from each of the terminal 200 of the user accompanied with the counseling recipient and the terminal 100 of the counseling recipient and obtain a drawing data for psychological counseling from the user terminal 100 or the user terminal 200, in order to obtain the drawing process that is not forged or falsified. The drawing process may be obtained after providing instruction data. The instruction data may be provided and the drawing process may be obtained from the terminal 100 of the counseling recipient, or the instruction data may be provided through the terminal 200 of the user accompanied with the counseling recipient and the drawing data may be obtained from the terminal 100 of the counseling recipient.

The management server 300 may be implemented to provide the instruction data through the terminal 100 of the counseling recipient or the terminal 200 of the user accompanied with the counseling recipient, and obtain the drawing data through the terminal 100 of the counseling recipient, but the present disclosure is not limited thereto, and various implementations are possible.

The management server 300 may obtain the drawing data for psychological counseling through the psychological counseling module 110 installed in the terminal 100 of the counseling recipient, and transmit the drawing data to the terminal 400 of the expert group to request analysis comments. The management server 300 may generate psychological state information of the counseling recipient by collecting the received analysis comments.

The management server 300 may transmit payment information corresponding to the provided analysis comments before providing the analysis comments on the drawing data for psychological counseling to the terminal 100 of the counseling recipient. The management server 300 may provide a certain reward to the terminal 400 of the expert group that has input the analysis comments. The reward provided to the terminal 400 of the expert group may be determined based on the number of input analysis comments and rating information for each analysis comment. The management server 300 may provide a reward proportional to the number of input analysis comments or a reward proportional to the rating for each analysis comment.

The management server 300 may store and manage the drawing data obtained from counseling recipients and the analysis comments on the drawing data in a separate database. The management server 300 may manage the drawing data and the analysis comments as a set, and may provide the drawing data and the analysis comments at a request of an expert or a user belonging to a study group. In this regard, the request may include processing payment information corresponding to the drawing data and the analysis comments. The payment information may be determined in proportion to the number of drawing data and analysis comments. In addition, the payment information may be changed according to the rating of a creator of each analysis comment.

The management server 300 may generate and manage rating information of a diagnostician who creates analysis comments. The rating information of the diagnostician may be calculated by synthesizing rating information about the analysis comments generated by the diagnostician. The rating information about the analysis comments may be determined by the counseling recipient of the analysis comments, an expert who has obtained and confirmed the analysis comments, or a study group.

The device described above may be implemented as a hardware component, a software component, and/or a combination of the hardware component and the software component. For example, the device and components described in the embodiments may be implemented using one or more general purpose or special purpose computers such as a processor, a controller, an ALU (arithmetic logic unit), a digital signal processor, a microcomputer, a FPGA (field programmable gate array), a PLU (programmable logic unit), a microprocessor, or any other device capable of executing and responding to instructions. The processing device may execute an operating system (OS) and one or more software applications running on the operating system. The processing device may also access, store, manipulate, process, and generate data in response to execution of the software. For convenience of understanding, although one processing device is sometimes described as being used, one of ordinary skill in the art will recognize that the processing device may include a plurality of processing elements and/or a plurality of types of processing elements. For example, the processing device may include a plurality of processors or one processor and one controller. Other processing configurations, such as parallel processors are also possible.

The software may include a computer program, code, instructions, or a combination of one or more of the foregoing, and may configure the processing device so that the processing device can operate as intended, or to independently or collectively give instructions to the processing device. The software and/or the data may be permanently or temporarily embodied in any type of machine, component, physical device, virtual equipment, computer storage media or devices, or transmitted signal waves, such that the software and/or the data is interpreted by the processing apparatus or provides an instruction or data to the processing device. The software may be distributed over a networked computer system and stored or executed in a distributed manner. The software and the data may be stored on one or more computer readable recording media.

The method according to the embodiment may be embodied in the form of program instructions that can be executed through various computer means and recorded in a computer-readable medium. The computer-readable medium may include program instructions, data files, data structures, and the like separately or in combinations. The program instructions to be recorded on the computer-readable recording medium may be specially designed and configured for the embodiments or may be well-known to and available to one of ordinary skill in the art of computer software. Examples of the computer-readable recording medium include magnetic media (e.g., hard disks, floppy disks, magnetic tapes, etc.), optical media (e.g., CD-ROMs, or DVDs), magneto-optical media (e.g., floptical disks), and hardware devices specifically configured to store and execute program instructions (e.g., ROM, RAM, flash memories, etc.) Examples of the program instructions are advanced language codes that can be executed by a computer by using an interpreter or the like as well as machine language codes made by a compiler. The hardware devices can be configured to function as one or more software modules so as to perform operations according to examples, or vice versa.

While the embodiments have been particularly shown and described with reference to examples thereof, it will be understood by those of ordinary skill in the art that various changes in form and details may be made therein without departing from the spirit and scope of the present disclosure as defined by the following claims. For example, an appropriate result may be attained even when the above-described techniques are performed in a different order from the above-described method, and/or components, such as the above-described system, structure, device, and circuit, are coupled or combined in a different form from the above-described methods or substituted for or replaced by other components or equivalents thereof.

Therefore, other implementations, other embodiments, and equivalents to the claims are also within the scope of the following claims.

1. A method of determining a psychological state of a counseling recipient through a drawing process, the method comprising:
receiving, by a user terminal, a psychological test start input with respect to the counseling recipient;
setting an area to be drawn in by the counseling recipient, in response to the psychological test start input;
outputting, by the user terminal, one or more pieces of instruction information in an order of registration, storing the area to be drawn in at a certain frame rate for a certain period of time from a time at which the instruction information is output, and obtaining drawing data corresponding to the one or more pieces of instruction information;
receiving, by the user terminal, a psychological test end input and sensing a final image in the area to be drawn in; and
transmitting, by the user terminal, the drawing data and the final image to a management server, and receiving analysis comments from a psychological counseling expert who has analyzed the drawing data and the final image from the management server.

2. The method of claim 1, further comprising:
selecting, by the management server receiving the drawing data and the final image, the drawing data and the final image, and selecting a psychological counseling expert group corresponding to the counseling recipient; and
transmitting a request for analysis of the drawing data and the final image to one or more electronic devices included in the psychological counseling expert group.

3. The method of claim 2, further comprising:
receiving, by the user terminal, analysis comments from the management server, and
outputting, through an output unit, detailed information comprising the analysis comments of the drawing data for each time and the number of received analysis comments which are included in an icon expressing the drawing data.

4. The method of claim 2, further comprising:
receiving, by the user terminal, analysis comments from the management server, and
classifying the analysis comments with respect to one or more objects included in the final image, and generating and providing information hierarchized in consideration of a relationship between the objects.

5. A computer program stored in a computer-readable storage medium for executing a method of determining a psychological state of a counseling recipient through a drawing process, the method comprising:
receiving, by a user terminal, a psychological test start input with respect to the counseling recipient;
setting an area to be drawn in by the counseling recipient, in response to the psychological test start input;
outputting, by the user terminal, one or more pieces of instruction information in an order of registration, storing the area to be drawn in at a certain frame rate for a certain period of time from a time at which the instruction information is output, and obtaining drawing data corresponding to the one or more pieces of instruction information;
receiving, by the user terminal, a psychological test end input and sensing a final image in the area to be drawn in; and
transmitting, by the user terminal, the drawing data and the final image to a management server, and receiving analysis comments from a psychological counseling expert who has analyzed the drawing data and the final image from the management server.
